(19) United States
(12) Patent Application Publication
(10) Pub. No.: US 2021/0318284 A1
(43) Pub. Date: Oct. 14, 2021

(54) RAPID RESPONSE CURVES AND SURVEY MEASUREMENTS

(71) Applicant: LI-COR, Inc., Lincoln, NE (US)

(72) Inventors: Patrick B. Morgan, Lincoln, NE (US); Dayle McDermitt, Lincoln, NE (US); Mark Johnson, Hickman, NE (US); Tom Avenson, Lincoln, NE (US); David Hanson, Albuquerque, NM (US); Doug Lynch, Lincoln, NE (US); Aaron Saathoff, Lincoln, NE (US)

(21) Appl. No.: 17/356,204

(22) Filed: Jun. 23, 2021

Related U.S. Application Data
(62) Division of application No. 15/811,210, filed on Nov. 13, 2017.
(60) Provisional application No. 62/423,668, filed on Nov. 17, 2016.

(51) Int. Cl. G01N 33/00

(52) U.S. Cl. CPC G01N 33/0098

(57) ABSTRACT

Systems and methods for measuring plant leaf gas exchange based on instantaneous mass balance in the sample chamber. The response of leaf net assimilation rate (Aₙₑₜ) to computed leaf internal CO₂ concentration (C₁) is measured by continuously varying the input CO₂ concentration and measuring the continuous difference between chamber input (reference) and output (sample) concentrations to compute a continuous series of Aₙₑₜ values, which can then be plotted against computed Cᵢ. When combined with a similar response test using an empty chamber test to allow for sample chamber mixing and/or gas analyzer match dynamics and/or small flow-related residual time delays, such method provides accurate and rapid A Cᵢ response (RACᵢR) curves in a much shorter time than conventional methods.

### CROSS-REFERENCE TO RELATED APPLICATIONS

This application is a divisional of U.S. application Ser. No. 15/811,210, which was filed on Nov. 13, 2017, titled “RAPID RESPONSE CURVES AND SURVEY MEASUREMENTS,” which claims priority to U.S. Provisional Application No. 62/423,668, filed on Nov. 17, 2016 and titled “RAPID RESPONSE CURVES AND SURVEY MEASUREMENTS.” Both applications are incorporated by reference in their entirety.

### BACKGROUND

Systems for measuring plant photosynthesis and transpiration rates can be categorized as open or closed systems. For open systems, the leaf or plant is enclosed in a sample chamber, and an air stream is passed continuously through the chamber. CO₂ and H₂O concentrations of chamber influent and effluent gasses are measured, and the difference between influent and effluent concentration is calculated. (Throughout this document the term “concentration” refers to mole fraction of a gas in natural or synthetic moist air, or mole fraction in natural or synthetic dry air (“dry mole fraction”) where such is specified.) This difference is used, along with the mass flow rate, to calculate photosynthesis (CO₂) and transpiration (H₂O) rates. For closed systems, the leaf or plant is enclosed in a chamber that is not supplied with fresh air. The concentrations of CO₂ and H₂O are continuously monitored within the chamber. The rate of change of this concentration, along with the chamber volume, is used to calculate photosynthesis (CO₂) and transpiration (H₂O) rates.

In both open and closed systems, it is important that the leaf or plant be the only source or sink of both CO₂ and H₂O. CO₂ or H₂O concentration changes not caused by the plant are a measurement error. These errors can be empirically compensated, for example as described in the LI-COR Biosciences LI-6400 User Manual (pp. 4-43 thru 4-48). Some instrument users may not understand the significance of these corrections, and neglect them.

Both open and closed systems contain a circuit of pneumatic components (e.g., pumps, valves, chambers, tubing, analyzers, etc.). When CO₂ and H₂O concentrations are dynamically changing, sorption on these components can provide an apparent CO₂ or H₂O source and/or sink. Under steady-state conditions, sorption is not an active source or sink, and persistent CO₂ and H₂O sources and/or sinks can be attributed to bulk leaks and diffusion.

In open photosynthesis systems, a conditioned air stream is typically split into two streams. The first flow path (known as reference) passes through a gas analyzer (e.g., Infra-Red Gas Analyzer or IRGA), which measures constituent gas concentrations (CO₂ and H₂O). The second flow path (known as sample) passes through a sample chamber (leaf chamber) in which gas exchange occurs. This second sample flow path exits the chamber and enters a second gas analyzer (e.g., IRGA) or alternates with the reference air stream through a single gas analyzer. The differences between the sample and reference gas concentrations are used in calculating photosynthesis (CO₂) and transpiration (H₂O). As photosynthesis and transpiration measurements are based on concentration differences in these two gas streams, the accuracy in measuring the difference is more critical than measuring the absolute concentration of either. Persistent diffusive sources and/or sinks present in the tubing, connectors, and fittings that supply the head with the sample and reference gas streams can compromise measurement accuracy.

The analytical method to measure photosynthetic CO₂ assimilation used over the past 40 years has been to provide a chamber input airstream with one, or a series of discrete values, of known and constant gas concentrations, and to allow the leaf to equilibrate to each new concentration. The assimilation rate is then measured, after steady state has been reached. The measurement requires the input concentration to be constant, and in the second case, requires time for the leaf to reach SS with the new concentration. This standard method works well but requires time and elaborate equipment.

### SUMMARY

The present disclosure provides systems and method for measuring plant leaf gas exchange based on instantaneous mass balance in the sample chamber. The new approach in the present embodiments includes applying analyses that exploit the ability to measure instantaneous mass balance in the leaf chamber due to the close proximity of gas flow components. This allows measurements with continuously variable gas concentration inputs that can be either controlled or uncontrolled.

According to an embodiment, a method is provided for determining a rapid net assimilation rate (Aₙₑₜ) to computed sample internal CO₂ concentration (Cᵢ) response (RACᵢR) curve for a photosynthesis capable sample in a gas exchange analysis system having an enclosed sample chamber defining a measurement volume for analysis of the photosynthesis capable sample, the sample chamber having an inlet port and an outlet port. The method typically includes, a) with the sample chamber empty, continuously varying a concentration of CO₂ introduced into a gas flow line connected with the inlet port of the sample chamber from a first concentration to a second concentration, and during the continuously varying: i) measuring, at each of a first plurality of measurement times, a first concentration of CO₂ in a gas exiting the sample chamber using a first gas analyzer, and ii) simultaneously measuring, at each of the first plurality of measurement times, a second concentration of CO₂ in the gas entering the sample chamber using a second gas analyzer, and iii) determining, for each of the first plurality of measurement times, an empty chamber assimilation rate value A_{EC} by subtracting the second concentration values from the first concentration values at each of the corresponding measurement times. The method also typically includes b) receiving a photosynthesis capable sample in the chamber, and c) with the photosynthesis capable sample in the chamber, continuously varying the concentration of CO₂ introduced into the gas line from the first concentration to the second concentration, and during the continuously varying: i) measuring, at each of a second plurality of measurement times, a third concentration of CO₂ in a gas exiting the sample chamber using the first gas analyzer, ii) simultaneously measuring, at each of the second plurality of measurement times, a fourth concentration of CO₂ in the gas entering the sample chamber using the second gas analyzer, and iii) determining, for each of the plurality of the second measurement times, an apparent assimilation rate value Aₐₚₚ by subtracting the fourth concentration values from the third concentration values at each of the corresponding measurement times. The method further typically includes d) determining a net assimilation rate value of the photosynthesis capable sample by subtracting the empty chamber assimilation value from the apparent assimilation value at each of the plurality of second measurement times.

In certain aspects, the concentration of CO₂ introduced into a gas flow line is continuously and linearly varied. In certain aspects, a non-linear or curved ramping technique is used, wherein the same non-linear or curved ramping technique is used for both the empty chamber and photosynthesis capable sample measurements. In certain aspects, the first plurality of measurement times have a same interval as the second plurality of measurement times. In certain aspects, steps b) and c) occur before step a). In certain aspects, steps b) and c) occur after step a). In certain aspects, the continuously and linearly varying the concentration of CO₂ includes only increasing the concentration of CO₂. In certain aspects, the continuously varying the concentration of CO₂ includes only decreasing the concentration of CO₂. In certain aspects, the continuously varying the concentration of CO₂ includes increasing then decreasing the concentration of CO₂, or decreasing then increasing the concentration of CO₂. In certain aspects, the photosynthesis capable sample includes a leaf or a whole plant. In certain aspects, the photosynthesis capable sample includes an organism such as cyanobacteria, euglena, algae, and anoxygenic photosynthesis bacteria.

In certain aspects, the determining the net assimilation rate value includes performing a correction where A_{EC}=f([CO₂]_{GA2}), with the function f parameterized to minimize A_{EC}, where GA2 refers to the second gas analyzer. In certain aspects, the determining the net assimilation rate value includes performing a linear regression on the empty chamber assimilation rate values, where

A_{EC}=m*[CO₂]_{GA2}−b, where GA2 refers to the second gas analyzer, m is the slope and b is a y-intercept. In certain aspects, the determining the net assimilation rate value includes performing a regression on the empty chamber assimilation rate values, where A_{EC}=a*[CO2]_{CA2}²+b*[CO2]_{GA2}+c, with a, b and c parameters from a 2^{nd} order polynomial. In certain aspects, the gas exchange analysis system includes a flow splitting mechanism located proximal to the sample chamber, and wherein the method further includes splitting a gas flow received from the gas flow line at an input port of the flow splitting mechanism to a first output port and to a second output port, wherein the first output port is coupled with the inlet port of the sample chamber, and wherein the second output port is coupled with the second gas analyzer.

According to another embodiment, a method is provided for determining a rapid net assimilation rate (Aₙₑₜ) to computed sample internal CO₂ concentration (Cᵢ) response (RACᵢR) curve for a photosynthesis capable sample in a gas exchange analysis system having an enclosed sample chamber defining a measurement volume for analysis of the photosynthesis capable sample, the sample chamber having an inlet port and an outlet port, and a flow splitting mechanism located proximal to the sample chamber. The method typically includes splitting a gas flow received from a gas flow line at an input port of the flow splitting mechanism to a first output port and to a second output port, wherein the first output port is coupled with the inlet port of the sample chamber, and with the sample chamber empty, continuously varying a concentration of CO₂ introduced into the gas line from a first concentration to a second concentration, and during the continuously varying: i) measuring, at each of a first plurality of measurement times, a first concentration of CO₂ in a gas exiting the outlet port of the sample chamber using a first gas analyzer; and ii) simultaneously measuring, at each of the first plurality of measurement times, a second concentration of CO₂ in the gas exiting the second output port of the flow splitting mechanism using a second gas analyzer; and iii) determining, for each of the first plurality of measurement times, an empty chamber assimilation rate value A_{EC} by subtracting the second concentration values from the first concentration values at each of the corresponding measurement times. The method also typically includes receiving a photosynthesis capable sample in the chamber, and with the photosynthesis capable sample in the chamber, continuously varying the concentration of CO₂ introduced into the gas line from the first concentration to the second concentration, and during the continuously varying: i) measuring, at each of a second plurality of measurement times, a third concentration of CO₂ in a gas exiting the outlet port of the sample chamber using the first gas analyzer, ii) simultaneously measuring, at each of the second plurality of measurement times, a fourth concentration of CO₂ in the gas exiting the second output port of the flow splitting mechanism using the second gas analyzer, and iii) determining, for each of the plurality of the second measurement times, an apparent assimilation rate value Aₐₚₚ by subtracting the fourth concentration values from the third concentration values at each of the corresponding measurement times. The method further typically includes determining a net assimilation rate value of the photosynthesis capable sample by subtracting the empty chamber assimilation value from the apparent assimilation value at each of the plurality of second measurement times.

According to a further embodiment, an open-path gas exchange analysis system for determining a rapid net assimilation rate (Aₙₑₜ) to computed sample internal CO₂ concentration (Cᵢ) response (RACᵢR) curve for a photosynthesis capable sample, is provided. The system typically includes a CO₂ source coupled to a gas flow line, wherein responsive to a received control signal, the CO₂ source adjusts a concentration of CO₂ provided to the gas flow line in a continuous and linear manner from a first concentration to a second concentration, an enclosed sample chamber having an inlet port and an outlet port, the inlet port coupled with the gas flow line, a first gas analyzer coupled to the outlet port of the enclosed sample chamber and configured to measure a first concentration of CO₂ exiting the enclosed sample chamber, a second gas analyzer coupled to the second output port of the flow splitting device and configured to measure a second concentration of CO₂ entering the enclosed sample chamber, and a control circuit. The control circuit typically is adapted to, or operates to: a) with the enclosed sample chamber empty, send a control signal to the CO₂ source to control the CO₂ source to continuously vary a concentration of CO₂ introduced into the gas line from the first concentration to the second concentration, and during the continuously varying: i) control the first gas analyzer to measure, at each of a first plurality of measurement times, a first concentration of CO₂ in a gas exiting the enclosed sample chamber, and ii) simultaneously control the second gas analyzer to measure, at each of the first plurality of measurement times, a second concentration of CO₂ in the gas entering the enclosed sample chamber, and iii) determine, for each of the first plurality of measurement times, an empty chamber assimilation rate value A_{EC} by subtracting the second concentration values from the first concentration values at each of the corresponding measurement times. The control circuit typically is adapted to, or operates to b) in response to an indication that a photosynthesis capable sample has been placed in the enclosed sample chamber, with the photosynthesis capable sample in the enclosed sample chamber, send a second control signal to the CO₂ source to control the CO₂ source to continuously vary the concentration of CO₂ introduced into the gas line from the first concentration to the second concentration, and during the continuously varying: i) control the first gas analyzer to measure, at each of a second plurality of measurement times, a third concentration of CO₂ in a gas exiting the enclosed sample chamber; ii) simultaneously control the second gas analyzer to measure, at each of the second plurality of measurement times, a fourth concentration of CO₂ in the gas entering the enclosed sample chamber, and iii) determine, for each of the plurality of the second measurement times, an apparent assimilation rate value Aₐₚₚ by subtracting the fourth concentration values from the third concentration values at each of the corresponding measurement times. The control circuit typically is adapted to, or operates to, c) determine a net assimilation rate value of the photosynthesis capable sample by subtracting the empty chamber assimilation value from the apparent assimilation value at each of the plurality of second measurement times.

According to yet another embodiment, an open-path gas exchange analysis system for determining a rapid net assimilation rate (Aₙₑₜ) to computed sample internal CO₂ concentration (Cᵢ) response (RACᵢR) curve for a photosynthesis capable sample is provided. The system typically includes a flow splitting device having an input port coupled to a gas flow line, a first output port and a second output port, the flow splitting device configured to split an incoming gas flow received from the gas flow line to the first and second output ports, a CO₂ source coupled to the gas flow line, wherein responsive to a received control signal, the CO₂ source adjusts a concentration of CO₂ provided to the gas flow line in a continuous manner from a first concentration to a second concentration, an enclosed sample chamber having an inlet port and an outlet port, the inlet port coupled with the first output port of the flow splitting device, a first gas analyzer coupled to the outlet port of the enclosed sample chamber and configured to measure a first concentration of CO₂ exiting the outlet port of the enclosed sample chamber, a second gas analyzer coupled to the second output port of the flow splitting device and configured to measure a second concentration of CO₂ exiting the second output port of the flow splitting device, and a control circuit. The control circuit typically is adapted to, or operates to: with the sample chamber empty, send a control signal to the CO₂ source to control the CO₂ source to continuously vary a concentration of CO₂ introduced into the gas line from the first concentration to the second concentration, and during the continuously varying: i) control the first gas analyzer to measure, at each of a first plurality of measurement times, a first concentration of CO₂ in a gas exiting the outlet port of the sample chamber, and ii) simultaneously control the second gas analyzer to measure, at each of the first plurality of measurement times, a second concentration of CO₂ in the gas exiting the second output port of the flow splitting mechanism, and iii) determine, for each of the first plurality of measurement times, an empty chamber assimilation rate value A_{EC} by subtracting the second concentration values from the first concentration values at each of the corresponding measurement times. The control circuit also typically is adapted to, or operates to, in response to an indication that a photosynthesis capable sample has been placed in the chamber, and with the photosynthesis capable sample in the chamber, send a second control signal to the CO₂ source to control the CO₂ source to continuously vary the concentration of CO₂ introduced into the gas line from the first concentration to the second concentration, and during the continuously varying: i) control the first gas analyzer to measure, at each of a second plurality of measurement times, a third concentration of CO₂ in a gas exiting the outlet port of the sample chamber, ii) simultaneously control the second gas analyzer to measure, at each of the second plurality of measurement times, a fourth concentration of CO₂ in the gas exiting the second output port of the flow splitting mechanism, and iii) determine, for each of the plurality of the second measurement times, an apparent assimilation rate value Aₐₚₚ by subtracting the fourth concentration values from the third concentration values at each of the corresponding measurement times. The control circuit also further typically is adapted to, or operates to, determine a net assimilation rate value of the photosynthesis capable sample by subtracting the empty chamber assimilation value from the apparent assimilation value at each of the plurality of second measurement times.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE SEVERAL VIEWS OF THE DRAWINGS

The detailed description is described with reference to the accompanying figures. The use of the same reference numbers in different instances in the description and the figures may indicate similar or identical items.

FIG. 1A shows an example data set showing an empty chamber test and Aₙₑₜ before correction.

FIG. 1B shows an example data set showing an empty chamber test and Aₙₑₜ after correction.

FIG. 2 shows ACᵢ curves prepared using corrected assimilation rates and the RACiR method (1), compared with traditional, steady-state measurements, according to an embodiment.

FIG. 3 illustrates a flow path in a photosynthesis measurement system according to an embodiment.

FIG. 4 illustrates a method of measuring a net assimilation rate value of a photosynthesis capable sample of a gas in a gas exchange analysis system according to one embodiment.

### DETAILED DESCRIPTION

The present disclosure provides systems and methods for measuring plant leaf gas exchange based upon instantaneous mass balance in a leaf chamber of a gas exchange measurement system.

The embodiments disclosed herein provide novel analytical systems and methods for measuring plant leaf gas exchange based upon instantaneous mass balance in the leaf sample chamber, due to the close physical proximity of the gas analyzer(s) to the points (i) where the incoming airflow is divided into sample and reference air flows, (ii) where the sample flow rate is measured and enters the leaf chamber, and/or (iii) where the sample flow leaves the leaf chamber. An example of a system incorporating such a physical layout is the LI-6800 Portable Photosynthesis System produced and sold by LI-COR Biosciences, Inc. The close physical proximity of the gas analyzers to the points (i) where the incoming airflow is divided into sample and reference air flows, (ii) where the sample flow rate is measured, and (iii) where the sample flow leaves the leaf chamber, makes it possible to perform a near instantaneous mass balance on gases entering and leaving the leaf chamber. This physical proximity is an important characteristic (1) allowing near instantaneous measurement of gas concentrations entering and leaving the leaf chamber and (2) for reducing diffusive sources and sinks. Examples of the physical layout and proximity of components are described in U.S. Pat. Nos. 8,610,072, 8,910,506, and 9,482,653, which are incorporated by reference in their entireties. In certain embodiments, the air flow leaving the chamber may be measured just outside the chamber, or it may be measured just inside the chamber. Similarly, air flow entering the chamber may be measured just outside the chamber, or it may be measured just inside the chamber.

The analytical method to measure photosynthetic CO₂ assimilation used over the past 40 years has been to provide a chamber input airstream with one, or a series of discrete values, of known and constant gas concentrations, and to allow the leaf to equilibrate to each new concentration. The assimilation rate is then measured, either over time as the leaf comes into steady state (SS) with the new concentration, or more commonly, after steady state has been reached. Both approaches require the input concentration to be constant, and in the second case, requires time for the leaf to reach SS with the new concentration. This standard method works well but requires time and elaborate equipment.

The new approach in the present embodiments includes applying analyses that exploit the ability to measure instantaneous mass balance in the leaf chamber due to the close proximity of components as mentioned above. This allows measurements with continuously variable gas concentration inputs that can be either controlled or uncontrolled. Two examples will illustrate the principles.

First, the response of leaf net assimilation rate (Aₙₑₜ) to computed leaf internal CO₂ concentration (Cᵢ) can be measured by continuously varying the input CO₂ concentration and measuring the continuous difference between chamber input (reference) and output (sample) concentrations to compute a continuous series of Aₙₑₜ values, which can then be plotted against computed Cᵢ. When combined with a similar response test using an empty chamber test to allow for sample chamber mixing and/or gas analyzer (e.g., IR gas analyzer or IRGA) match dynamics and/or small flow-related residual time delays, this method provides accurate and rapid A Cᵢ response (RACᵢR) curves in a much shorter time than conventional methods (5-10 min vs 30-60 min) as will be discussed in more detail below. This is termed herein the RACᵢR method. The RACᵢR method is advantageous because it allows rapid measurement of important plant biochemical features (e.g. V_{cmax}, carboxylation efficiency (CE), Jₘₐₓ, and others) in a shorter time than prior methodologies while holding other chamber environmental conditions constant. This capability is important for large-scale screening of plant phenotypes, for example. The RACᵢR method has the potential to be faster than some biological processes, like stomatal closure or enzyme activation, thereby removing or reducing their impact on the measurement. The RACᵢR method is possible and practical because the close proximity of system components, such as in the design of the LI-6800, allows instantaneous estimates of leaf chamber inputs and outputs with high temporal fidelity. This is non-intuitive for even experienced users because the general belief is that the time required for conventional (non-RACiR) methods is needed to achieve the steady state biochemistry required for models of photosynthesis, which has been shown not to be true in a number of important cases.

Second, given instantaneous mass balance, average Aₙₑₜ can be measured in an open gas exchange system when the input CO₂ concentration is uncontrolled and variable in time, for example as supplied by the ambient atmosphere; or when output CO₂ concentration varies, for example because a change in light intensity caused Aₙₑₜ to vary; or when both occur in any combination. The idea is that one knows what goes into the chamber and what comes out on a near instantaneous basis over a given time interval (Δt), and how the chamber CO₂ concentration changes over Δt, so those values can be integrated over Δt and the average Aₙₑₜ computed. This is termed herein the Integration Method. The Integration Method is advantageous because it allows in-the-field Aₙₑₜ measurements without requiring a complicated air supply console that can provide a fixed and constant incoming CO₂ concentration. Over the years that field-portable open photosynthesis systems have been available, one of the central problems for those systems has been the need to supply an air input with constant CO₂ concentration. The embodiments herein solve that problem. For example, in certain embodiments, the air supply unit need only supply ambient air, and it need not fix or control the gas concentrations of that air, making the device simpler, more portable, and less expensive. It will be obvious to one skilled in the art that similar comments apply to other instrument environmental control systems, including but not limited to light or temperature control systems.

In certain device embodiments, the air flow is split between sample and reference paths in the measurement head, e.g., immediately before the flow meter, sample chamber and gas analyzers (GAs), so times required for flows to transport chamber input and output gas concentrations to the GAs are much shorter than in other portable gas exchange systems. This makes it possible to measure a nearly instantaneous mass balance in the sample chamber. The reference and sample GAs report gas concentrations entering and leaving the leaf chamber with excellent temporal fidelity because flow rate-dependent time delays are quite small (e.g., ˜500 ms at normal flow rates).

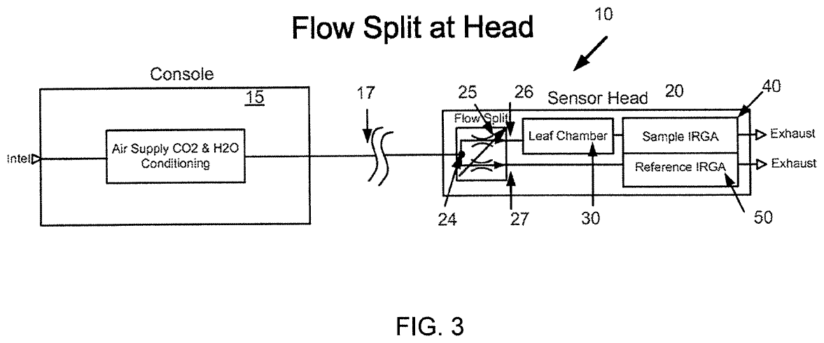

FIG. 3 illustrates a flow path in an exemplary gas exchange measurement system 10 according to one embodiment. Gas exchange measurement system 10 in one embodiment includes a console 15 and a sensor head 20 remote from console 15. Other system embodiments contemplate an integrated console and sensor head or sensor module. Console 15 typically includes, or is connected with, one or more gas sources and gas conditioning equipment. For example, in the context of photosynthesis and transpiration measurements, gas sources would include reservoirs of CO₂ and H₂O, and conditioning equipment for controlling and conditioning each gas concentration in a gas flow line. A flow path or gas flow line 17 connecting console 15 with sensor head 20 typically includes flexible tubing and connectors. Flow path 17 provides a single stream or gas flow path to flow splitting device or mechanism 25 in sensor head 20. Flow splitting device or mechanism 25 receives a stream of gas from console 15 and splits the flow into two separate flow paths as will be described in more detail below. One stream is provided to the chamber 30 (e.g., sample stream) and the other stream (e.g., reference stream) is provided to a reference gas analyzer 50. A second gas analyzer 40 receives and analyzes gas exiting from chamber 30. Reference gas analyzer 50 and second gas analyzer 40 might each include an Infra-Red Gas Analyzer (IRGA), as is known in the art, or other gas analyzer.

It is desirable that flow path lengths and the number of connections downstream of the flow split device or mechanism 25 location be minimized to reduce parasitic sources and sinks which differentially affect concentrations in the two flow paths. Hence, according to one embodiment, the flow path is split in the sensor head proximal to the sample chamber. The majority of parasitic sources and sinks, which are located upstream of the sensor head in FIG. 3, affect only a single air stream (flow path 17) when the flow is split at the sensor head 20. Parasitic sources and sinks which impact the sample and reference streams independently are advantageously minimized.

It is desirable that for a certain flow rate, through either the reference or sample path, less than a certain amount of diffusion occurs. Therefore, according to one embodiment, the flow is split as close to the sample chamber and gas analyzers as possible. In certain aspects, the flow splitting device or mechanism 25 is located such that a minimal amount of flow path having components or surface areas exposed or susceptible to diffusion exists between the flow splitting device 25 and the sample chamber 30. The desired length of the flow path is generally a function of the flow rate and the diffusion susceptible material or components making up the flow path; for example, for metal tubing, the flow path can be significantly longer than for plastic or other diffusion-susceptible components. For example, in certain aspects, a flow path having 12″ or less of diffusion-susceptible tubing and/or other components is desirable between the flow splitting device or mechanism 25 and the sample chamber 30 to provide a gas stream flow path from the splitting device or mechanism 25. In other aspects, less than about 6″, or 4″ or 2″ or even 1″ or less of such diffusion-susceptible flow path exists between the flow splitting device or mechanism 25 and the sample chamber 30.

Similarly, in certain aspects, the flow splitting mechanism is located in the sensor 30 head such that less than about 12″ of such diffusion-susceptible flow path exists between the flow splitting device or mechanism 25 and the reference gas analyzer 50. In other aspects, the flow splitting device or mechanism is located such that less than about 6″, or 4″ or 2″ or even 1″ or less of such flow path exists between the flow splitting device or mechanism 25 and the reference gas analyzer 50. It is also desirable that that flow path length between the sample chamber 30 and sample gas analyzer 40 be minimized. One skilled in the art will appreciate that the diffusion-susceptible flow path from the flow splitting device or mechanism 25 to the reference gas analyzer 50 can be roughly the same length as the diffusion-susceptible flow path from the splitting device or mechanism 25 through the sample chamber 30 to the sample gas analyzer 40. Alternately, the two diffusion susceptible flow paths can be different lengths as desired.

For the RACiR method, when incoming CO₂ concentration is continuously increased (or decreased), the increase (or decrease) will be measured immediately by the reference GA 50, but the sample GA 40 will see a delayed output because the sample chamber acts as a mixing volume diluting the increase with a first-order time constant given, approximately, by chamber volume divided by volumetric flow rate (e.g., typically near 5s). Chamber mixing will be complete after three to five time constants and then, if the chamber is empty, CO₂ concentration in the chamber will increase at the same rate as the input CO₂ concentration, although its value will be offset in time. A similar delay will occur if a sample (e.g., leaf or other photosynthesis capable sample) is present in the chamber but the steady rate of increase that follows will reflect the difference between the CO₂ input rate and the rate of CO₂ removal (or addition) by the leaf. Measured values for apparent Aₙₑₜ are determined by the instantaneous CO₂ concentration difference measured between sample GA and reference GAs which is due to the sum of four contributions: (1) uptake of CO₂ by a sample, if present, (2) the amount by which the chamber CO₂ concentration lags the incoming reference CO₂ concentration due to volumetric mixing and dilution in the chamber, (3) small GA match offsets that may accumulate as the reference CO₂ concentration increases (or decreases), and (4) any small residual errors due to flow-related time delays in transporting air to the GAs. The last three contributors arise from properties of the system and are the same with or without a sample in the sample chamber so they can be measured in an empty chamber test.

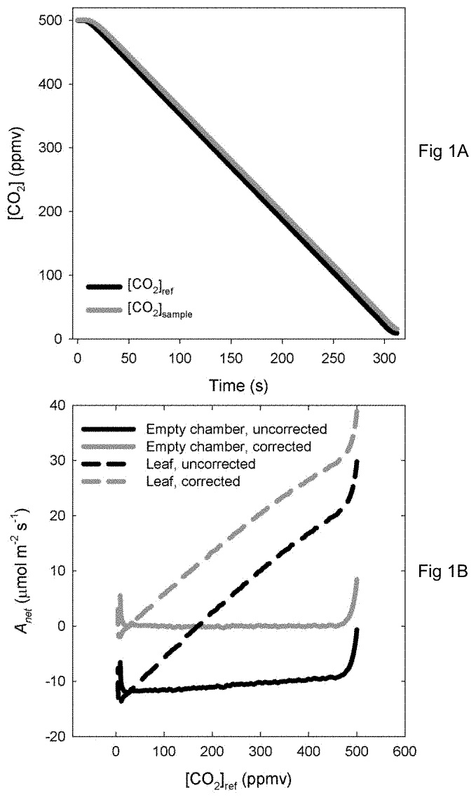

For RACiR measurements, data can be analyzed in either of two ways: (1) an empirical method in which Aₙₑₜ measured point-by-point as chamber and reference CO₂ concentrations increase (or decrease) is corrected by subtracting corresponding apparent Aₙₑₜ values obtained from an empty chamber test with the same flow rates (FIGS. 1A and 1B). The correction is obtained in two steps: first, using data obtained with an empty chamber, a regression is performed over an appropriate range (e.g., linear range) of apparent reference Aₙₑₜ vs reference CO₂ concentration. This range may be linear or slightly variable. In the latter case a polynomial regression may be used. The resulting equation computes corrected reference Aₙₑₜ as a function of reference CO₂ concentration. Second, corrected Aₙₑₜ values are then obtained by subtracting corrected reference Aₙₑₜ point-by-point from Aₙₑₜ values measured with a leaf in the chamber at corresponding CO₂ concentrations. This will correct all of the last three contributions mentioned above. Example data sets showing an empty chamber test and Aₙₑₜ before and after correction are shown in FIGS. 1A and 1B.

In an embodiment, in both the empty chamber response test and the sample-filed chamber test, the CO₂ concentration is linearly and continuously ramped (increased or decreased). For example, the concentration may be ramped from a starting value of 0 μmolmol⁻¹ or a higher value to about 300 μmol mol⁻¹ or 500 μmol mol⁻¹ or 1000 μmol mol⁻¹ or greater to a greater value, or the CO₂ concentration may be ramped from a starting value of about 1000 μmol mol⁻¹ or greater or smaller down to 0 μmol mol⁻¹ or down to an intermediate value. The rate of attenuation or increase may be controlled as desired, for example 100 μmol mol⁻¹ mini, or greater or smaller, e.g., between 1 μmol mol⁻¹ mini and 2000 μmol mol⁻¹ mini. The ramping may be linear, e.g., continuous and linear, or the ramping may take on a non-linear curved shape. In an embodiment, there are no “pauses” in the CO₂ ramping. However, introducing brief pauses into the ramp is contemplated, but would slow down the measurement process.

(2) The second analysis involves performing an analytical mass balance based upon the difference between sample and reference concentrations and the rate of change of chamber dry CO₂ concentration. Preliminary experiments with an empty chamber show such corrections can be readily applied. The chamber mass balance is given by

$$A = \frac{u}{s} ( {C}_{e} - {C}_{o} ) - \frac{V ρ}{s} \frac{d {C}_{o}}{d t} \qquad equation 1$$

where Cₑ and Cₒ are dry CO₂ mole fractions (herein referred to as “concentrations”, Cᵢ=Cᵢ(moist)/(1−wᵢ), where wᵢ is mole fraction of water vapor) entering and leaving the leaf chamber, respectively. With perfect mixing, Cₒ equals the chamber concentration. This does not require an empty chamber test to be paired with each sample measurement, but it does require that dCₒ/dt is computed from the chamber concentration time course and additional consideration must be given to small time delay and GA match offsets. Time delay offsets are due to small differences in length of the sample and reference flow paths. Match offsets are the result of very small differences in response of the sample and reference GAs as CO₂ concentration changes; both are small and fixed so they can be estimated in advance with empty chamber tests.

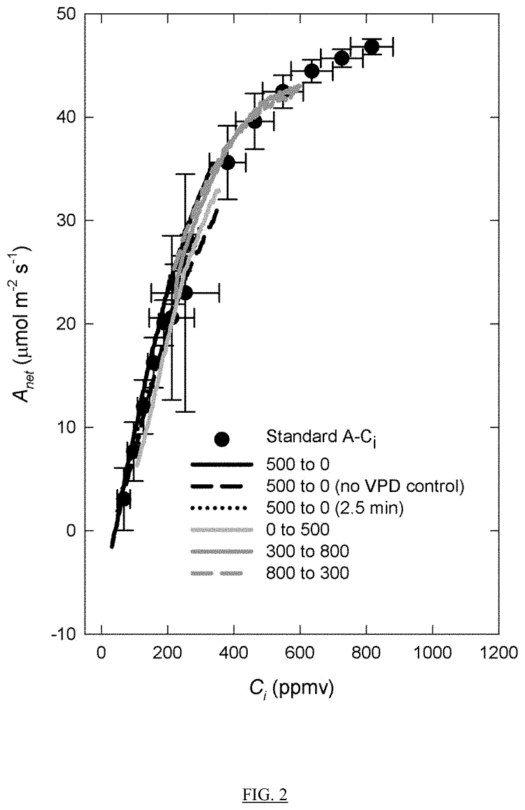

FIG. 2 shows ACᵢ curves prepared using corrected assimilation rates and the RACiR method (1), compared with traditional, steady-state measurements. Results using the RACiR method are quite similar to those obtained with the traditional method, but were obtained in less than half the time.

The Integration Method also requires a chamber mass balance. But here the goal is not to produce ACᵢ curves, but rather to compute average Aₙₑₜ when the incoming airstream has variable or uncontrolled CO₂ concentration, such as one would obtain using the ambient atmosphere as CO₂ source, or when the assimilation rate itself is variable for one reason or another, e.g., variations in other environmental variables such as temperature, light intensity, etc. It can be shown that average Aₙₑₜ measured over an interval Δt is given by

$$\overline{A} = \frac{u}{s} ( \overline{{C}_{e}} - \overline{{C}_{o}} ) - \frac{V ρ}{s} \frac{Δ {C}_{o}}{Δ t} \qquad equation 2$$

where the average values are computed over Δt and ΔCₒ=Cₒ (initial)−Cₒ(final) is the change in chamber CO₂ dry mole fraction over the interval Δt. The second term on the right gives the change in CO₂ storage in the leaf chamber over Δt. The Integration Method is advantageously easy to apply but it has important implications for instrument simplicity, as described above.

In certain embodiments, the Integration Method may be used in conditions where incoming CO₂ is controlled but sample CO₂ is rapidly changed through alteration of the sample environment and the effects on the biochemistry of the enclosed tissue changes the rate of net CO₂ exchange. For example, rapid changes in the light intensity cause photosynthesis to change sample CO₂ rapidly while reference CO₂ is held constant. This allows for other rapid response measurements like RACiR to be conducted, but where environmental variables besides CO₂ concentration are changed rapidly.

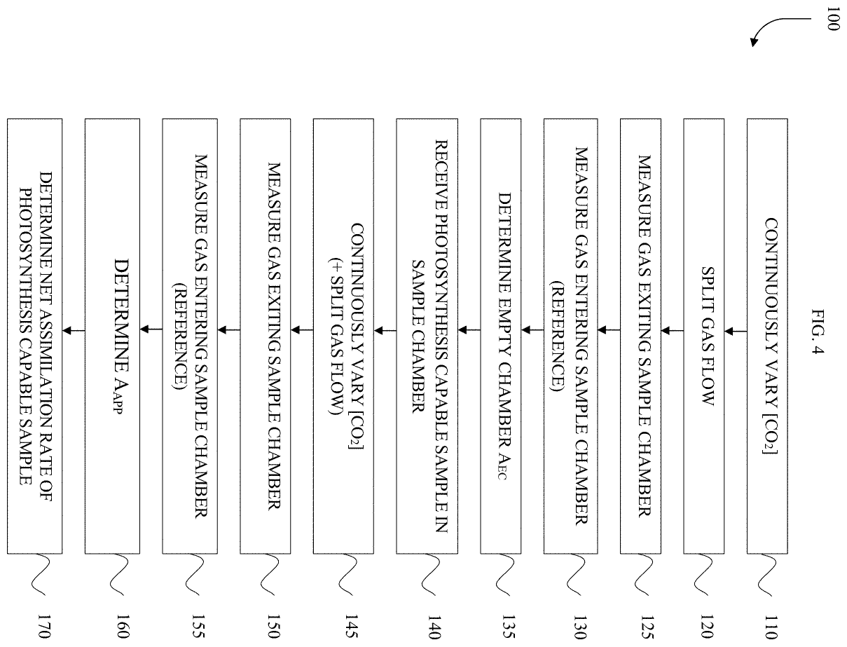

FIG. 4 illustrates a method 100 of measuring a net assimilation rate value of a photosynthesis capable sample of a gas in a gas exchange analysis system according to one embodiment. The gas exchange analysis system in certain embodiments includes a flow splitting device or mechanism located proximal to a sample chamber that defines a measurement volume for analysis of a sample. The sample chamber includes an inlet and an outlet, with the inlet being connected, in close proximity, with an output (e.g., port) of the flow splitting device. The outlet is connected, also preferably in close proximity, with a gas analyzer such as an IRGA. In step 110, a concentration of CO₂ introduced into a gas flow line connected with the inlet port of the sample chamber is continuously varied from a first concentration to a second concentration. As the CO₂ concentration is continuously varied, in step 120, a gas flow received from the gas flow line at an input port of the flow splitting mechanism is controllably split to a first output port and to a second output port, with the first output port being coupled with the inlet of the sample chamber. In step 125, with the sample chamber empty, during the continuously varying of the CO₂ concentration, a first concentration of one or more gases exiting the sample chamber is measured using a first gas analyzer (e.g., gas analyzer 40) fluidly coupled with an output of the sample chamber. For example, at each of a first plurality of measurement times, a first concentration of CO₂ in a gas exiting the sample chamber is measured using the first gas analyzer 40. Similarly, in step 130, during the continuously varying of the CO₂ concentration, a second concentration of the one or more gases exiting the second output port is measured using a second gas analyzer (e.g., gas analyzer 50) fluidly coupled with the second output port of the flow splitting device. For example, at each of the first plurality of measurement times, a second concentration of CO₂ in the gas entering the empty sample chamber is measured using the second gas analyzer 50. In step 135, for each of the first plurality of measurement times, an empty chamber assimilation rate value A_{EC} by subtracting the second concentration values from the first concentration values at each of the corresponding measurement times.

In step 140, a sample, e.g., photosynthesis capable material or substance, is received in the sample chamber. In step 145, the concentration of CO₂ introduced into the gas flow line connected with the inlet port of the sample chamber is continuously varied from the first concentration to the second concentration. In step 150, the with the sample chamber containing the sample, as the CO₂ concentration is continuously varied, a third concentration of one or more gases exiting the sample chamber is measured using the first gas analyzer (e.g., gas analyzer 40) fluidly coupled with an output of the sample chamber. For example, at each of a second plurality of measurement times, a third concentration of CO₂ in a gas exiting the sample-filled sample chamber is measured using the first gas analyzer 40. Similarly, in step 155, during the continuously varying of the CO₂ concentration, a fourth concentration of the one or more gases exiting the second output port is measured using the second gas analyzer (e.g., gas analyzer 50) fluidly coupled with the second output port of the flow splitting device. For example, at each of the second plurality of measurement times, a fourth concentration of CO₂ in the gas entering the sample-filled sample chamber is measured using the second gas analyzer 50. In step 160, for each of the second plurality of measurement times, an apparent assimilation rate value Aₐₚₚ is determined by subtracting the fourth concentration values from the third concentration values at each of the corresponding measurement times. It should be appreciated that the empty chamber measurements of steps 110-130 may be performed before or after the sample-filled chamber measurements of steps 145-155. It should also be appreciated that the first and second plurality of measurement times may be the same or different, e.g., the same or different time intervals between measurements.

In step 170, a net assimilation rate value of the photosynthesis capable sample is determined by subtracting the empty chamber assimilation value from the apparent assimilation value, e.g., at each of the plurality of second measurement times. Steps 135, 160 and 170 can be performed using a processing component, e.g., processor or computer system, that is integrated in the sensor head and/or in the console of the gas analysis system and/or in a remote computer system that is communicably coupled with the gas analysis system. In step 180, the net assimilation rate value is output, e.g., displayed on a monitor or other output device, printed, stored, or otherwise provided to another computer system or device. Other determined data values may also be output as desired.

In some embodiments, a flow slitting mechanism may not be present, e.g., gas is sampled before entering the sample chamber and after entering the sample chamber.

In some instruments, the relationship between Aₐₚₚₐᵣₑₙₜ and reference [CO₂] in an empty chamber (equation 1) may be non-linear. In those instances, a higher order polynomial fit may be needed to make the corrections, but the results are otherwise unchanged. For an individual instrument the extent and shape of any non-linearity may be influenced by the CO₂ mole fraction of the gas chosen to set the span. In those cases, the equation may take the form A_{EC}=e[CO2]_{GA2}²+b*[CO2]_{GA2}+c, with a, b and c parameters from a 2^{nd} order polynomial. However, any equation will suffice as long as A_{EC} is some function of [CO2]_{GA2} that minimizes the values of A_{EC}.

For example, the net assimilation rate value may be determined by performing a correction of the empty chamber Assimilation rates where A_{EC}=f([CO2]_{GA2}), with the function f parameterized to minimize A_{EC}.

In certain embodiments, an intelligence module, including a processing component such as one or more processors and associated memory and/or storage, is coupled with the gas analyzer and the flow control system components and is adapted to control operation of such components and to receive and process data from such components to implement the methods disclosed herein, e.g., perform the RACᵢR calculations and store received and processed data. For example, the processing component may include a processor or control circuit that sends one or more control signals to the CO₂ source to control the CO₂ source to continuously and linearly vary a concentration of CO₂ introduced into the gas line from a first concentration to a second concentration.

The processing component is configured to implement functionality and/or process instructions for execution, for example, instructions stored in memory or instructions stored on storage devices. The processing component may be implemented as an ASIC including an integrated instruction set. The memory, which may be a non-transient computer-readable storage medium, is configured to store information during operation. In some embodiments, the memory includes a temporary memory, area for information not to be maintained when the processing component is turned OFF. Examples of such temporary memory include volatile memories such as random access memories (RAM), dynamic random access memories (DRAM), and static random access memories (SRAM). The memory maintains program instructions for execution by the processing component. Example programs can include the RACiR methodology and the Integration methodology described herein.

Storage devices also include one or more non-transient computer-readable storage media. Storage devices are generally configured to store larger amounts of information than the memory. Storage devices may further be configured for long-term storage of information. In some examples, storage devices include non-volatile storage elements. Non-limiting examples of non-volatile storage elements include magnetic hard disks, optical discs, floppy discs, flash memories, or forms of electrically programmable memories (EPROM) or electrically erasable and programmable (EEPROM) memories.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the disclosed subject matter (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or example language (e.g., “such as”) provided herein, is intended merely to better illuminate the disclosed subject matter and does not pose a limitation on the scope of the invention unless otherwise claimed. No language in the specification should be construed as indicating any non-claimed element as essential to the practice of the invention.

Certain embodiments are described herein. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. For example, the methodologies disclosed herein may be useful to determine response to other gases, or components in a gas, such as H₂O, O₂, etc. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A method for determining a rapid net assimilation rate (Aₙₑₜ) to computed sample internal CO₂ concentration (Cᵢ) response (RACᵢR) curve for a photosynthesis capable sample in a gas exchange analysis system having an enclosed sample chamber defining a measurement volume, V, for analysis of the photosynthesis capable sample, the sample chamber having an inlet port and an outlet port, the method comprising:
with a photosynthesis capable sample in the sample chamber, continuously varying one or more of a) a concentration of CO₂ introduced into a gas flow line connected with the inlet port of the sample chamber from a first concentration to a second concentration, b) an intensity of light illuminating the photosynthesis capable sample in the sample chamber, and c) a temperature of the measurement volume of the sample chamber, during the continuously varying:
i) measuring, at each of a first plurality of measurement times, a first concentration of CO₂ in a gas exiting the sample chamber using the first gas analyzer;
ii) measuring, at each of the first plurality of measurement times, a second concentration of CO₂ in the gas entering the sample chamber using the second gas analyzer; and
iii) determining, for each of the first plurality of measurement times, an apparent assimilation rate value Aₐₚₚ, wherein the determining the apparent assimilation rate value Aₐₚₚ includes subtracting the measured first concentration value from the measured second concentration value;
determining a net assimilation rate value of the photosynthesis capable sample by solving a chamber mass balance equation for the sample chamber; and
providing the net assimilation rate value as an output.

2. The method of claim 23, wherein i) and ii) are performed simultaneously.

3. The method of claim 23, wherein the chamber mass balance equation solved for assimilation is given by
$$A = \frac{u}{s} ( {C}_{e} - {C}_{o} ) - \frac{V ρ}{s} \frac{d {C}_{o}}{d t}$$
where Cₑ and Cₒ are dry CO₂ mole fractions entering and leaving the sample chamber, respectively, where ρ is the density of the air in the sample chamber, u is the air mass flow rate, and s is an area of the sample in the sample chamber.

4. An open-path gas exchange analysis system for determining a rapid net assimilation rate (Aₙₑₜ) to computed sample internal CO₂ concentration (Cᵢ) response (RACᵢR) curve for a photosynthesis capable sample, the system comprising:
a CO₂ source coupled to a gas flow line, wherein responsive to a received control signal, the CO₂ source adjusts a concentration of CO₂ provided to the gas flow line in a continuous manner from a first concentration to a second concentration;
an enclosed sample chamber having an inlet port and an outlet port, the inlet port coupled with the gas flow line;
a first gas analyzer coupled to the outlet port of the enclosed sample chamber and configured to measure a first concentration of CO₂ exiting the enclosed sample chamber;
a second gas analyzer coupled to the second output port of the flow splitting device and configured to measure a second concentration of CO₂ entering the enclosed sample chamber; and
a control circuit, the control circuit adapted to:
a) in response to an indication that a photosynthesis capable sample has been placed in the enclosed sample chamber:
with the photosynthesis capable sample in the enclosed sample chamber, send a second control signal to the CO₂ source to control the CO₂ source to continuously vary the concentration of CO₂ introduced into the gas flow line from the first concentration to the second concentration, and during the continuously varying:
i) control the first gas analyzer to measure, at each of a first plurality of measurement times, a first concentration of CO₂ in a gas exiting the enclosed sample chamber;
ii) control the second gas analyzer to measure, at each of the first plurality of measurement times, a second concentration of CO₂ in the gas entering the enclosed sample chamber;
iii) determine, for each of the plurality of the first measurement times, an apparent assimilation rate value Aₐₚₚ, wherein the determining the apparent assimilation rate value Aₐₚₚ includes subtracting the first concentration value from the second concentration value; and
c) determine a net assimilation rate value of the photosynthesis capable sample by solving a chamber mass balance equation for the sample chamber.

5. The system of claim 26, wherein the control circuit is adapted to perform i) and ii) simultaneously.

6. The system of claim 26, wherein the chamber mass balance equation solved for assimilation is given by
$$A = \frac{u}{s} ( {C}_{e} - {C}_{o} ) - \frac{V ρ}{s} \frac{d {C}_{o}}{d t} ,$$
where Cₑ and Cₒ are dry CO₂ mole fractions entering and leaving the sample chamber, respectively, ρ is the density of the air in the sample chamber and s is an area of the sample in the sample chamber.

7. A method for determining a rapid net water vapor transpiration rate (Eₜ) for a photosynthesis capable sample in a gas exchange analysis system having an enclosed sample chamber defining a measurement volume, V, for analysis of the photosynthesis capable sample, the sample chamber having an inlet port and an outlet port, the method comprising:
with a photosynthesis capable sample in the sample chamber, continuously varying one or more of a) a concentration of CO₂ introduced into a gas flow line connected with the inlet port of the sample chamber from a first concentration to a second concentration, b) an intensity of light illuminating the photosynthesis capable sample in the sample chamber, and c) a temperature of the measurement volume of the sample chamber, during the continuously varying:
i) measuring, at each of a first plurality of measurement times, a first concentration of H₂O in a gas exiting the sample chamber using the first gas analyzer;
ii) measuring, at each of the first plurality of measurement times, a second concentration of H₂O in the gas entering the sample chamber using the second gas analyzer; and
iii) determining, for each of the first plurality of measurement times, an apparent transpiration rate value Eₐₚₚ, wherein the determining the apparent transpiration rate value Eₐₚₚ includes subtracting the measured second concentration value from the measured first concentration value;
determining a net water vapor transpiration rate value E of the photosynthesis capable sample by solving a chamber mass balance equation for the sample chamber; and
providing the net transpiration rate value as an output.

8. A method for determining an average net assimilation rate (Aₙₑₜ) for a photosynthesis capable sample in a gas exchange analysis system having an enclosed sample chamber defining a measurement volume for analysis of the photosynthesis capable sample, the sample chamber having an inlet port and an outlet port, the method comprising:
with a photosynthesis capable sample in the sample chamber:
i) measuring, at each of a first plurality of measurement times, a first concentration of CO₂ in a gas exiting the sample chamber using the first gas analyzer;
ii) measuring, at each of the first plurality of measurement times, a second concentration of CO₂ in the gas entering the sample chamber using the first gas analyzer or a second gas analyzer; and
iii) determining, for each of the first plurality of measurement times, an apparent assimilation rate value Aₐₚₚ, wherein the determining the apparent assimilation rate value Aₐₚₚ includes subtracting the measured first concentration value from the measured second concentration value;
determining an average net assimilation rate value of the photosynthesis capable sample by solving a chamber mass balance equation for the sample chamber; and
providing the average net assimilation rate value as an output.

9. The method of claim 8, further including, with the photosynthesis capable sample in the sample chamber, varying one or more of a) a concentration of CO₂ introduced into a gas flow line connected with the inlet port of the sample chamber from a first concentration to a second concentration, b) an intensity of light illuminating the photosynthesis capable sample in the sample chamber, and c) a temperature of the measurement volume of the sample chamber, during the varying.
